(19) United States
(12) Patent Application Publication
(10) Pub. No.: US 2008/0171070 A1
(43) Pub. Date: Jul. 17, 2008

(54) Polyelectrolyte Multilayer Film, Preparation And Uses Thereof

(76) Inventors: Pierre Schaaf, Molsheim (FR); Nadia Jessel, Strasbourg (FR); Joelle Ogier-Dirrig, Strasbourg (FR); Philippe Lavalle, Strasbourg (FR); Jean-Claude Voegel, Valff (FR); Bernard Senger, Gimbrett (FR)

Correspondence Address: NIXON & VANDERHYE, PC, 901 NORTH GLEBE ROAD, 11TH FLOOR, ARLINGTON, VA, 22203, US

(21) Appl. No.: 11/883,375

(22) PCT Filed: Jan. 31, 2006
(86) PCT No.: PCT/IB06/00378

§ 371 Date: Jul. 31, 2007

Related U.S. Application Data
(60) Provisional application No. 60/648,279, filed on Jan. 31, 2005.

(51) Int. Cl. A61L 27/54; B05D 1/02; B05D 1/18; B05D 1/28; A61F 2/06; B05D 1/40; B05D 1/42

(52) U.S. Cl. 424/422; 427/2.24; 623/1.42; 514/12

(57) ABSTRACT

The invention relates to polyelectrolyte multilayer films presenting a tunable biological activity and methods for preparing the same. The invention further relates to surfaces presenting such films and uses thereof, such as for the controlled delivery of biologically active agents.

### FIELD OF THE INVENTION

The invention relates to polyelectrolyte multilayer films presenting a tunable biological activity and methods for preparing the same. The invention further relates to surfaces presenting such films and uses thereof, such as for the controlled delivery of biologically active agents.

### BACKGROUND OF THE INVENTION

Over the past years, great efforts were devoted to render materials biologically active. The first trials were aimed at providing them with a single functionality. For example, coronary stents coated with heparin present antithrombotic properties^{[1]} or coated with an anti-proliferative agent, such as rapamycin, reduce restenosis.^{[2]} Different methods were developed to this aim: active molecules were incorporated directly into the material^{[3-5]} or were fixed on the surface of the material merely by adsorption^{[6,7,8]} or by chemical grafting.^{[9-11]} Bioactive molecules, such as insulin^{[12]} or epidermal growth factor^{[13]}, have, for example, been chemically grafted and immobilized on surfaces. None of these methods is, however, free of drawbacks: the incorporation of active molecules into the bulk of a given material is not always possible; adsorption of molecules often involves weak bonds so that the molecules rapidly desorb and chemical grafting can be very difficult to achieve. Moreover, the irreversible attachment of molecules to a surface may also sometimes reduce their biological activity. A second generation of bioactive materials presenting time scheduled activity and multifunctionalization is now under development. Very recently, Langer and coworkers presented biodegradable polymeric microchips that release pulses of active molecules within a precision of a few days over a period of five months.^{[14]} These chips are constituted of macroscopic reservoirs filled with the active molecules and closed by a biodegradable membrane constituted of poly(d, l-lactic-co-glycolic acid).

The deposition of polyelectrolyte multilayers on charged surfaces offers a new alternative solution to functionalize biomaterials.^{[15]} These coatings are obtained by the alternate dipping of a charged surface in polyanion and polycation solutions. The simplicity and versatility of such a build-up procedure opens great perspectives for its widespread use in biomaterial coating. The inventors have recently equipped polyelectrolyte multilayers with anti-inflammatory properties by incorporating anti-inflammatory drugs or peptides into the film architectures.^{[16-18]} Bioactive proteins can also be directly integrated in the architecture without any covalent bonding with a polyelectrolyte and keep a secondary structure close to that of their native form.^{[19-25]} Partially degradable layered structures could thus be advantageous for progressive delivery of associated active agents.

Recently, the inventors demonstrated that cells were able to react with protein A (PA) embedded in (PlGA/PlL) multilayer architectures.^{[19]} This protein issued from the cell wall of Staphylococcus aureus possesses the ability to bind the Fc fragment of IgG and has also a large panel of biological activities: it is an antitumoral,^{[26,27]} antitoxic,^{[28]} anticarcinogenic,^{[29]} antifungal^{[30]} and antiparasitic agent.^{[31]} Besides, PA stimulation of the human macrophages leads to the rapid expression of both the pro-inflammatory cytokine TNF-α and the anti-inflammatory cytokine IL-10.^{[32]} The inventors examined the effect of the embedding depth of PA in (PlGA/PlL)ₙ multilayers on its activity by measuring the amount of TNF-α produced by cells grown on these films. The inventors found that cells interact with PA incorporated in polyelectrolyte multilayer films and showed that they come in contact with the active protein by degrading the film. Values of TNF-α production obtained after 4 hours and one night of cell interaction with the films were similar whatever the embedding depth of PA (up to n=30) and were comparable to the value obtained when PA was adsorbed on the terminating layer. Finally, the inventors have shown that the replacement of PlL by PdL forms a barrier that prevents cellular communication with embedded PA.^{[19]}

It was also recently demonstrated that polyelectrolyte multilayers can be built by using polyanion or polycation mixtures instead of one component solutions.^{[33-36]} In this case, the two polyelectrolytes from a mixture are incorporated simultaneously into the multilayer during each deposition step. This leads to new film properties which lie between the extreme properties obtained with the one-component polyelectrolyte solutions. These properties can be tuned by changing the mixing ratio of the polyelectrolytes in the mixture.^{[35,36]}

It would be desirable to provide systems having controllable or adjustable release properties. In particular a method would be favourable which allows the release of materials into and from systems by modifying permeability thereof. Further, for most applications a defined and controllable permeability of the system is required in order to control the process of release under specific environmental conditions.

Using the fact that the embedding of protein A in a film composed of d polypeptide enantiomers extinguishes completely the biological activity of the film whereas full activity is obtained with l enantiomers, the inventors have discovered herein that the biological activity of the film can be tuned in time by using poly(lysine)/poly(glutamic acid) multilayers constructed with polyanion and polycation solutions each constituted of d and l mixtures with different d/l content ratios.

### SUMMARY OF THE INVENTION

It is therefore an object of the invention to provide polyelectrolyte multilayers films presenting a controllable and defined delivery of active ingredients included therein.

It is a further object of the invention to provide a method for producing such polyelectrolyte multilayer films and in particular a method for coating a surface with such polyelectrolyte multilayer films and the coated article obtained there from.

This invention demonstrates the possibility to tune the biological activity of a surface functionalized by polyelectrolyte multilayers. Protein A interacting with macrophages is used as a model system, but the results could have been obtained with other kinds of active ingredients. The film may be constituted by two polypeptides, poly(lysine) and poly(glutamic acid), each build-up solution being a mixture of the respective l- and d-enantiomers of either poly(lysine) or poly(glutamic acid). Cells are deposited on top of the film and produce TNF-α as they enter into contact with the protein.

Depending upon the d/l-enantiomer rate (or d percentage) of the polyelectrolyte solutions used for the film buildup and the embedding depth of the protein, the production of TNF-α sets on after a varying, controllable, and adjustable induction time and displays a transition from no-production to full-production taking place over a lapse time which also depends on the film composition and embedding depth. Thus, it is shown that changing these two parameters permits an accurate tuning of the protein activity in time.

### LEGENDS TO THE FIGURES

FIG. 1: Observation by confocal laser scanning microscopy: a) phagocytosis of PLL^{FITC} by the cell b) Pseudopods formation by the cell. Both frame a) and frame b) correspond to 20 min of contact with the multilayer film containing PA embedded under 20 (PlGA-PlL) pairs of layers. c) Cells in contact with a multilayer film containing the PA^{TR} (TR: Texas red) embedded under 20 (PlGA-PlL) pairs of layers after overnight (15 hours) contact. Image size=48.7×48.7 μm².

FIG. 2: Surface structure of a multilayer film containing PA embedded under 20 (PlGA/PlL) pairs of layers after incubation with cells and observed by confocal laser scanning microscopy (a: 0 min, b: 180 min, c: 15 h (overnight)). The terminating layer was formed by FITC conjugated PlL. (d): Surface structure of a multilayer film containing PA embedded under 20 (PdGA/PdL) pairs of layers after incubation with cells overnight.

FIG. 3: The main frame shows the variation of the thickness of the films, d, as new layers are added (PL stands for poly(lysine) and PGA for poly(glutamic acid)) for various values, x, of the d content (0% (◯), 10% (∇), 30% (□), 50% (⋄), 100% (Δ). The insert shows the thickness of (PL/PGA)₆ films as a function of x.

FIG. 4: TNF-α secretion by macrophages grown on polyelectrolyte films. Cells were incubated for 1, 2, 3, 4 and 6 hours. The height of each bar corresponds to the optical density (OD) at 450 nm wavelength averaged over two independent experiments. The error bars represent the standard deviation. The different polyelectrolyte film architectures correspond to n=0: [(PlL/PlGA)₅-PlL-PA], n=1: [(PlL/PlGA)₅-PlL-PA-(PL/PGA)-PL], n=5: [(PlL/PlGA)₅-PlL-PA-(PL/PGA)₅-PL], n=15: [(PlL/PlGA)₅-PlL-PA-(PL/PGA)₁₅-PL], and n=20: [(PlL/PlGA)₅-PlL-PA-(PL/PGA)₂₀-PL]. The contributions of the PlL, PdL, PlGA and PdGA of the enantiomers of PL and PGA in the layers constituting the upper part of the films are specified by the value of x indicated in each frame. x: 100% (A), x: 50% (B), x: 40% (C), x: 30% (D), x: 20% (E), x: 10% (F).

FIG. 5: FTIR spectra measured in the transmission mode in the peptide amide-1 region (maximum at 1658 cm⁻¹) PA (◯); PdL (▪); PlL (□); Complex of PdL and PA (♦); Complex of PlL and PA (⋄); Sum of PA and PdL spectra (▴); Sum of PA and PlL spectra (Δ).

### DETAILED DESCRIPTION OF THE INVENTION

According to a first aspect, the invention deals with a polyelectrolyte multilayer film, wherein said film comprises at least one layer pair of cationic polypeptides and anionic polypeptides, and wherein said cationic polypeptides comprise l and d amino-acid forms and said anionic polypeptides comprise l and d amino-acid forms.

In a particular embodiment, the polyelectrolyte multilayer film further comprises at least one positively and/or negatively charged biologically active ingredient.

Said active ingredient can be embedded at any depth of the film of the invention. The depth of the active ingredient is determined by one skilled in the art as to obtain the desired results, including the desired release time and release amount of the active ingredient.

The cationic polypeptides are any polypeptide having cationic (e.g. cationically dissociable) groups. They are more preferably selected in the group consisting of poly(lysine), poly(arginine), poly(omithine), poly(histidine) and mixtures thereof or more generally of any kind of l and d forms of cationic polypeptides.

The anionic polypeptides are any polypeptide having anionic (e.g. anionically dissociable) groups. They are more preferably are selected in the group consisting of poly(glutamic acid), poly(aspartic acid) and mixtures thereof or more generally of any kind of l and d forms of anionic polypeptides.

In a preferred embodiment, the cationic polypeptides and anionic polypeptides are respectively poly(lysine) and poly(glutamic acid).

According to a particular embodiment of the invention, the polyelectrolyte multilayers can further comprise different types of polymers with different functional groups, including cationic polymers (sulfonium, phosphonium, ammonium, hydroxylamine, hydrazide such as poly(hydroxylamine), poly(hydrazide), poly(diallydimethylammonium chloride), poly(allylamine), poly(ethylene)imine, chitosan, poly(mannoseamine), and other sugars), anionic polymers (including poly(acrylic) acid, poly(methacrylic) acid, poly(styrene sulfonate), poly(phosphate), polynucleic acid, polyuronic acid (alginic, galacturonic, glucuronic, etc), glycosaminoglycans (hyaluronic acid, also called hyaluronan, dermatan sulphate, chondroitin sulphate, heparin, heparan sulphate, and keratan sulphate), etc., neutral polymers (including polyacrylamide, polyethylene oxyde, polyvinyl alcohol), and mixtures thereof.

The molecular weight of the polymers identified above can vary in a wide range. More preferably, the molecular weight is in the range from 0.5 kDa to 20,000 kDa, even more preferably, the molecular weight is in the range from 5 to 2,000 kDa.

The positively and/or negatively charged biologically active ingredient can be a large variety of materials, including synthetic polyions (polymers presenting ions), biopolymers such as DNA, RNA, collagen, peptides (such as a RGD sequence, Melanoma stimulating Hormone, or buforin), proteins, growth factors, and enzymes, cells, viruses, dendrimers, colloids, inorganic and organic particles, dyes, vesicles, nano(or micro)capsules, nano(or micro)particles, polyelectrolytes complexes, free or complexed drugs, cyclodextrins, and more generally any object of interest for biological applications and mixtures thereof, may be readily incorporated into the polyelectrolyte multilayers. Said incorporation is well known in the art and can easily be carried out by one of ordinary skill in the art. In particular, said ingredients may be incorporated by adsorption or diffusion, or by coupling said materials to at least one of polyelectrolytes and adsorption thereafter of said polyelectrolyte.

The polyelectrolyte multilayers films and the coated article of the invention comprising such active ingredient are of particular interest, since such materials comprised therein keep their functions and/or activities, as stated above and illustrated by the examples.

An advantage of the method for preparing the films according to the invention is that the incorporation of the active ingredient can be performed at very well defined depths in the film with a precision of a few tens of nanometers, and in specific amounts. This advantage allows to control the release time and amount of the active ingredient.

Moreover, by using polyelectrolytes that are degradable and non-degradable (d and l forms), the release of the active ingredient can be controlled based on the rate of degradability of the polyelectrolyte layers. As used herein, a “degradable” material is a material which undergoes dissolution, resorption and/or other degradation processes upon administration to a patient.

The film according to the invention generally presents a number of layer pairs from 1 to 1000, preferably from 2 to 100, more preferably from 5 to 60.

The film according to the invention may further comprise other types of polyelectrolyte multilayer films beneath or on the film as described hereinbefore. The active ingredient can be incorporated at any level of the film, including in the film as described hereinbefore and/or other types of polyelectrolyte multilayer films.

In a particular embodiment, the film according to the invention comprises (1) a first polyelectrolyte multilayer film, said first film (or precursor film) comprising at least one positively and/or negatively charged biologically active ingredient as defined above and at least one, preferably five, layer pair of cationic polypeptides and anionic polypeptides, said polypeptides presenting only l amino-acid form, and (2) a second polyelectrolyte multilayer film as described above comprising at least one layer pair of cationic polypeptides and anionic polypeptides and wherein each cationic or anionic polypeptide layer comprises l and d amino-acid forms.

Due to the identical chemical nature of the used l and d amino-acid forms, the inventors could not determine in which proportion they are incorporated in the film. In the present description, we shall assume, without loss of generality, that the two enantiomers of each polypeptide are incorporated in the film in the same proportion as in the build-up polypeptide solutions.

To that respect, the percentage of l and d amino-acid forms in each cationic or anionic polypeptide layer may vary in a large extent and will depend directly from the choice made by one skilled in the art when preparing the film according to the invention. This choice will depend upon the desired results and could be determined upon experimental assays.

According to a particular embodiment, the percentage of l and d amino-acid forms in the cationic polypeptide layer is the same as the percentage of l and d amino-acid forms in the anionic polypeptide layer.

The film according to invention, and more preferably the polyelectrolyte multilayer film comprising d and l amino-acid forms, the weight percentage x % of d amino-acid form present in the polypeptides of the multilayer film (preferably the second polyelectrolyte multilayer film as identified above) is from 0.1 to 50%, more preferably from 10 to 40%, and more preferably from 20 to 40%.

The x % of d enantiomer is the weight percentage of the total amount of d amino-acid form/total amount of d and l amino-acid forms in the polypeptides.

The first film according to the preceding embodiment may present a number of layer pairs in the first film from 1 to 1000, preferably from 2 to 100, and more preferably from 5 to 60.

The second film according to the preceding embodiment may present a number of layer pairs in the second film from 1 to 100, preferably from 2 to 100, and more preferably from 20 to 60.

According to another aspect, the invention provides a method of coating a surface, wherein said method comprises (1) sequentially depositing on a surface alternating layers of polyelectrolytes to provide a coated surface, wherein a first (or conversely second) polymer is a cationic polypeptide and a second (or conversely first) polymer is an anionic polypolypeptide, said cationic polypeptides comprise l and d amino-acid forms and said anionic polypeptides comprise l and d amino-acid forms.

The method according to the invention advantageously further comprises (2) reacting a surface with a solution comprising at least one positively and/or negatively charged biologically active ingredient. More specifically, said reaction allows to get said ingredient adsorbed onto the surface.

Said step (2) may be carried out before or after step (1). When step (1) is performed before step (2), step (2) is performed on the coated surface obtained by step (1). According to this embodiment, the method may further comprise, after step (2), an additional step (1), and optionally implementation of additional step(s) (2) and/or step(s) (1).

According to a particular embodiment, the surface, before step (1), is a surface coated by a first film (or precursor film) comprising at least one layer pair of cationic polypeptides and anionic polypeptides, said polypeptides presenting only l amino-acid forms, and optionally at least one positively and/or negatively charged biologically active ingredient.

The surface to be coated can be a portion of the surface or the whole surface of the article such as defined above.

Sequentially depositing on a surface alternating layers of polyelectrolytes may be accomplished in a number of ways, including dipping, dip-coating, rinsing, dip-rinsing, spraying, inkjet printing, stamping, printing and microcontact printing, wiping, doctor blading or spin coating.

Depositing on a surface alternating layers of polypolypeptides includes more particularly coating and rinsing steps.

Another coating process embodiment involves solely spray-coating and spray-rinsing steps. However, a number of alternatives involves various combinations of spray- and dip-coating and rinsing steps.

These methods may be designed by a person having ordinary skill in the art in accordance to the contemplated properties of the coated surface.

One dip-coating alternative involves the steps of applying a coating of a first polyelectrolyte to a surface by immersing said surface in a first solution of a first polyelectrolyte; rinsing the surface by immersing the surface in a rinsing solution; and, optionally, drying said surface. This procedure is then repeated using a second polyelectrolyte, with the second polyelectrolyte having charges opposite of the charges of the first polyelectrolyte, in order to form a polyelectrolyte pair of layers.

This layer pairs formation process may be repeated a plurality of times in order to produce a thicker surface coating. A preferred number of layer pairs is about 1 to about 1000. A more preferred number of layer pairs is about 5 to about 60.

In a particular embodiment, the thickness of the film is from 20 nm to 150 μm.

The immersion time for each of the coating and rinsing steps may vary depending on a number of factors. Preferably, contact times of the surface into the polyelectrolyte solution occurs over a period of about 1 second to 30 minutes, more preferably about 1 to 20 minutes, and most preferably about 1 to 15 minutes. Rinsing may be accomplished in one step, but a plurality of rinsing steps has been found to be quite efficient. Rinsing in a series of about 2 to 5 steps is preferred, with contact times with the rinsing solution preferably consuming about 1 to about 6 minutes.

Another embodiment of the coating process involves a series of spray coating techniques. The process generally includes the steps of applying a coating of a first polyelectrolyte to a surface by contacting the surface with a first solution of a first polyelectrolyte; rinsing the surface by spraying the surface with a rinsing solution; and, optionally, drying the surface. Similar to the dip-coating process, the spray-coating process may then be repeated with a second polyelectrolyte, with the second polyelectrolyte having charges opposite of the charges of the first polyelectrolyte.

The contacting of surface with solution, either polyelectrolyte or rinsing solution, may occur by a variety of methods. For example, the surface may be dipped into both solutions. One preferred alternative is to apply the solutions in a spray or mist form. Of course, various combinations may be envisioned, e.g., dipping the surface in the polyelectrolyte followed by spraying the rinsing solution.

The spray coating application may be accomplished via a number of methods known in the art. For example, a conventional spray coating arrangement may be used, i.e., the liquid material is sprayed by application of fluid, which may or may not be at elevated pressure, through a reduced diameter nozzle which is directed towards the deposition target.

Suitable solvents for polyelectrolyte solutions and rinsing solutions are: water, aqueous solutions of salts (for example NaCl, MnCl₂, (NH₄)₂ SO₄), any type of physiological buffer (Hepes, phosphate buffer, culture medium such as minimum essential medium, Mes-Tris buffer) and water-miscible, non-ionic solvents, such as C1-C4-alkanols, C3-C6-ketones including cyclohexanone, tetrahydrofuran, dioxane, dimethyl sulphoxide, ethylene glycol, propylene glycol and oligomers of ethylene glycol and propylene glycol and ethers thereof and open-chain and cyclic amides, such as dimethylformamide, dimethylacetamide, N-methylpyrrolidone and others. Polar, water-immiscible solvents, such as chloroform or methylene chloride, which can contain a portion of the abovementioned organic solvents, insofar as they are miscible with them, will only be considered in special cases. Water or solvent mixtures, one component of which is water, are preferably used. If permitted by the solubility of the polyelectrolytes implemented, only water is used as the solvent, since this simplifies the process.

The present invention also relates to the coated article obtained by the method as described above.

The coated article is selected from the group consisting of blood vessel stents, angioplasty balloons, vascular graft tubing, prosthetic blood vessels, vascular shunts, heart valves, artificial heart components, pacemakers, pacemaker electrodes, pacemaker leads, ventricular assist devices, contact lenses, intraocular lenses, sponges for tissue engineering, foams for tissue engineering, matrices for tissue engineering, scaffolds for tissue engineering, biomedical membranes, dialysis membranes, cell-encapsulating membranes, drug delivery reservoirs, drug delivery matrices, drug delivery pumps, catheters, tubing, cosmetic surgery prostheses, orthopaedic prostheses, dental prostheses, bone and dental implant, wound dressings, sutures, soft tissue repair meshes, percutaneous devices, diagnostic biosensors, cellular arrays, cellular networks, microfluidic devices, and protein arrays.

Further aspects and advantages of the present invention will be disclosed in the following examples, which should be regarded as illustrative and not limiting the scope of this application. All cited references are incorporated therein by references.

### EXAMPLES

Chemicals. Poly(L-lysine) hydrobromide (PlL, MW=39,000 Da), poly(L-lysine) hydrobromide labelled with fluorescein isothiocyanate (PlL^{FITC}, MW=23,000 Da), poly-(D-lysine) (PdL, MW=28,000 Da, Sigma), poly(D-glutamic acid) (PdGA, MW=44,700 Da), poly (L-glutamic acid) (PlGA, MW=53,785 Da) were purchased from Sigma and used without any further purification. The degree of substitution of PlL^{FITC} is 7 mmol FITC per lysine monomer. The Staphylococcus aureus protein A and PA labeled by sulforhodamine 101 acid chloride (Texas Red) (PA, MW=42,000 Da) was from Sigma (Ref. P7837).

Cell Culture. Whole blood samples were purchased at the “Etablissement Français du Sang” (EFS, Strasbourg, France). Peripheral mononuclear blood cells (PBMC) from healthy individuals, seronegative for HUV-1 and hepatitis B and C were isolated from buffy coat by Ficoll/Hypaque centrifugation and were washed twice in phosphate-buffered saline without Ca²⁺/Mg²⁺. Monocytes were isolated from whole-blood and separated by counter-current centrifugal elutriation of the peripheral mononuclear cells.^{[40]} Purity was measured by flow cytometry staining with fluorochrome antibodies (Becton Dickinson, PharMingen, San Diego, Calif.) to CD3 (T cells), CD19 (B cells), CD14 (monocytes) and CD45 (leukocytes). Monocytes were diluted at 1.5×10⁶ cells mL⁻¹ in AIM lymphocytes SVF free medium with Glutamax, 100 U mL⁻¹ GM-CSF (PeproTech, Rocky Hill, USA). Culture medium was changed after 3 days of culture, and at day 5 macrophages were washed twice with RPMI at 37° C.

Polyelectrolyte multilayered film preparation. Polyelectrolyte multilayers were always prepared on glass coverslips (CML, France) pretreated for 15 min at 100° C. with 10⁻² M SDS and 0.12 N HCl, and then extensively rinsed with deionised water. Glass coverslips were deposited in 24-well plates (Nunc, Denmark). All the solutions (polyelectrolyte, PA and rinsing) used for the film constructions contained 0.15 M NaCl with a pH adjusted to 7.4. At this pH both polylysine and polyglutamic acid are almost fully charged, be they l or d enantiomers so that they should not form stereocomplexes in solution. The films were constructed with polyelectrolyte (resp. PA) solutions at 1 mg·mL⁻¹ (resp. 200 μg·mL⁻¹) of polyelectrolytes (resp. PA). The film construction was performed as follows: First a precursor film constituted by (PlL/PlGA)₅-PlL was built. In each deposition step, the surface is brought in contact with the polyelectrolyte solution for 20 mins followed by another contact with the rinsing solution for 5 mins. This rinsing step is repeated 3 times before adsorption of the polyelectrolyte of opposite charge. After the buildup of the precursor film, PA was adsorbed on the positively charged PlL terminating the precursor film during an overnight contact with PA solution. This film was then rinsed and the additional (PL/PGA)ₙ-PL film was built by using PL and PGA solutions containing (x/100) mg·mL⁻¹ of PdL (resp. PdGA) and (1-x/100) mg·mL⁻¹ of PlL (resp. PlGA). All the films were then sterilized for 10 mins by ultraviolet light (254 nm). Before use, the architectures were put in contact with 1 mL of RPMI without serum during 24 h. It is possible that when the films are brought in contact with the culture medium, their structure and thickness change, the structure of the multilayers being dependent on many different physico-chemical parameters.^{[41-43]} However, such changes cannot explain the different biological effects that are observed in this study. Indeed, it is expected that the structure of PlL/PlGA multilayers would change in a similar way as would the structure of PdL/PdGA films but, as it is shown in this study, the former films show strong biological activity whereas the latter ones do not. One can also notice that the films were built at room temperature whereas the experiments with cells were realized at 37° C. Previous studies by Boulmedais et al.³⁸ showed that temperature changes from 20 to 37° C. did not affect the secondary structure of PlL/PGA multilayers and should thus also not greatly affect the structure of our films.

Stimulation assays. Stimulation assays involving polyelectrolyte films were conducted by seeding 5×10⁵ cells (macrophages) onto the PA-containing polyelectrolyte multilayers prepared on glass coverslips and placed into the wells of 24-well plates. TNF-α production by cells was measured by ELISA. TNF-α levels were detected by an enzyme immunoassay (Endogen Products, Woburn, Mass.). All experiments were repeated twice and were performed at 37° C.

Confocal laser scanning microscopy. For the confocal laser scanning microscopy (CLSM) based investigations, the films were imaged in liquid conditions. CLSM observations were carried out with a Zeiss LSM 510 microscope using a ×40/1.4 oil immersion objective and with 0.4 μm z-section intervals. FITC fluorescence was detected after excitation at 488 nm, cutoff dichroic mirror 488 nm, and emission band pass filter 505-530 nm (green). Virtual vertical sections can be visualized, allowing the thickness of the film to be determined.

Quartz crystal microbalance with dissipation. The quartz crystal microbalance with dissipation (QCMD, Q-Sense, Göteborg, Sweden) allows the recording of the resonance frequencies of a quartz crystal when a film is deposited on it. In addition, it permits the dissipation to be measured which is representative of the damping of the crystal oscillations once the excitation electric tension is switched off. Both the resonance frequencies and the dissipation depend on the thickness and the viscoelastic properties of the deposited film. The variation of thickness of the films along their buildup can be derived from these measurements by processing them with the viscoelastic model developed by Voinova et al.^{[37]}

FTIR Spectroscopy in transmission mode. Transmission spectra were measured on an EQUINOX 55 spectrophotometer (Bruker, Wissembourg, France) using a DTGS detector. Solutions were flown into a sample cell holder (SPECAC P/N 20510). Transmission spectra were measured by using CaF₂ windows. Single channel spectra from 128 interferograms were calculated between 4000 and 400 cm⁻¹ with 2 cm⁻¹ resolution, using Blackman-Harris three-term apodization and Mertz phase correction with the standard Bruker OPUS/IR software (Version 3.0.4). Each sample (aqueous solution of polypeptide or protein A or both) was prepared at a concentration of 1 mg mL⁻¹ of each component, in a D₂O, 0.15M NaCl (purchased from Prolabo) solution.

### Results and Discussion

### 1. Confocal Microscopy Observations

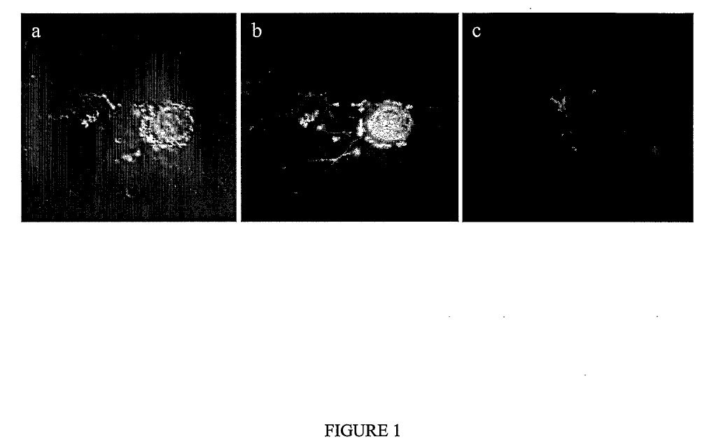

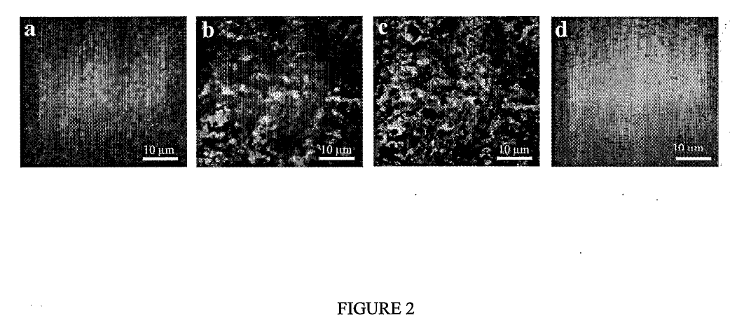

Confocal laser scanning microscopy (CLSM) allowed the inventors to demonstrate that the macrophages develop pseudopods along the film and that they come into contact with protein A even though it is embedded under 20 pairs of (PlGA/PlL) layers (FIG. 1). In previous experiments, the inventors could visualize the pseudopods developing through the multilayer down to the protein A layer.^{[19]} The direct interaction of protein A and the cells is further demonstrated here by the fact that the cell becomes red due to the Texas-Red labeled protein A (FIG. 1c). The progressive degradation of the (PlGA/PlL)ₙ film as a function of contact time with the cells is visualized in FIGS. 2a-2c. One observes, in particular, the presence of holes in the (PlGA/PlL)ₙ film whereas no holes are found in the (PdGA/PdL)ₙ film after one night of contact of the film with cells (FIG. 2d).

### 2. Buildup of Multilayer Films

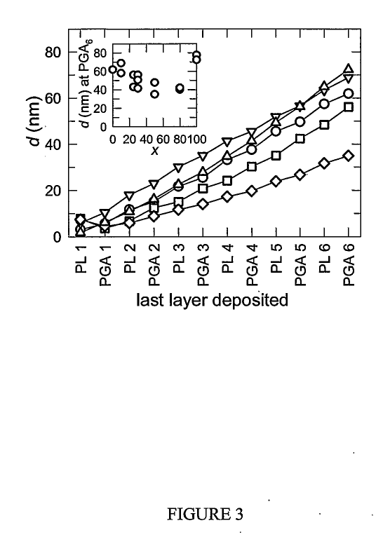

The inventors used poly-l-glutamic acid (PlGA) and poly-l-lysine (PlL) as degradable polyelectrolytes and poly-d-glutamic acid (PdGA) and poly-d-lysine (PdL) as non-degradable polyelectrolytes. The (poly(lysine)/poly(glutamic acid))ₙ films were grown by using PdGA/PlGA and PdL/PlL mixtures containing similar d/l ratios. These ratios were varied from one construction to another. The protein A molecules were embedded at different depths inside these architectures. The multilayered films were constructed by dipping a glass coverslip alternatively into the poly(lysine) and poly(glutamic acid) solutions containing the appropriate amounts of the d and l forms of the polyelectrolytes. The total polyanion and polycation concentration was kept fixed at 1 mg mL⁻¹ and the x % of d enantiomer was varied from 0 up to 100%. A film corresponding to x % of d was thus constructed using the mixture of a poly(lysine) solution containing x % of PdL and (100-x) % of PlL with a poly(glutamic acid) solution containing similarly x % of PdGA and (100-x) % of PlGA. The inventors first verified that the multilayer buildup was possible for any value of x between 0 and 100%. To this end, film constructions corresponding to different x values were followed by quartz crystal microbalance with dissipation (QCM-D). The inventors always found a steady decrease of the shifts of the measured quartz resonance frequencies with the number of deposited bilayers which proves the continuous film growth. Treating the data by the viscoelastic model developed by Voinova et al^{[37]} the inventors could determine the increase of the film thickness d (nm), as the build-up process went on (FIG. 3, main frame). The insert shows the thickness reached by a (PL/PGA)₆ film as a function of x. One observes that the film thickness depends on the d content of the build-up solutions. The thickness is approximately symmetric with respect to x=50% where it goes through a minimum. The change in the film buildup with the l/d ratio shows that the l enantiomers interact differently with l and d enantiomers of the polypeptide of opposite charge. This was already found by Boulmedais et al.^{[38]} who investigated the construction of PdL/PlGA multilayers. They found that the thickness of this film increases more slowly with the number of deposition steps than that of PlL/PlGA multilayers. This indicates that PdL interacts with PlGA but that their interactions are weaker than those of PlL with PlGA. It is thus also expected that the interactions of PlL with PdGA are weaker than that of PdL with PdGA. This could explain the minimum in the film thickness observed for the l/d ratio close to 50%. Indeed the difference in the interactions between l and d enantiomers of polypeptides of opposite sign should lead to some segregation on the surface between l and d polylysine/polyglutamic acid complexes. Such a segregation could lead to a decrease of the film thickness. At x=50% 50%, the segregation would be maximum if all the chains would have the same mass and mass distribution and would thus also lead to a minimum in the film thickness.

### 3. Cell Activity

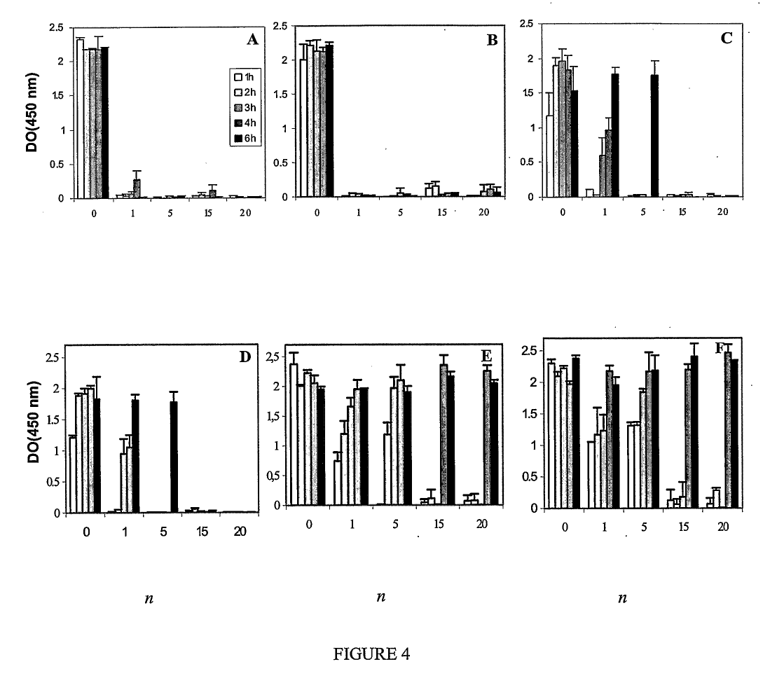

For multilayer films containing embedded PA, the proteins were always adsorbed on a (PlL/PlGA)₅-PlL precursor film in order to keep the adsorption process (in particular the amount of adsorbed proteins) unaffected by the d/l composition of the film. The multilayer with a given d/l composition was then further deposited on top of the (PlL/PlGA)₅-PlL-PA architecture and is constituted by n additional pairs of layers. The inventors always ended the architecture with a poly(lysine) layer in order to promote cell adhesion.^{[39]} These films were then brought in contact with human macrophages for interaction times ranging from 1 up to 6 hours. The biological activity will be followed by measuring TNF-α production. It was compared to the TNF-α production corresponding to a similar film which does not contain protein A. Substracting the second value of TNF-α production from the first one gives the additional activity due to the presence of protein A. These results are gathered in FIG. 4.

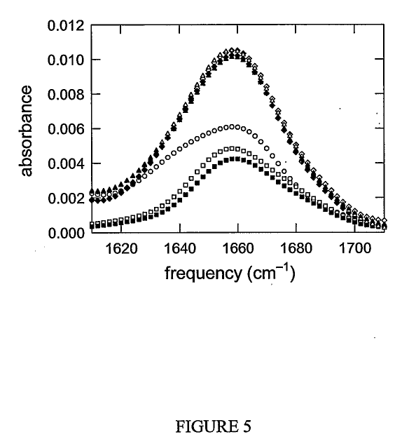

First, one can notice the good experimental reproducibility for the experiments in which PA is adsorbed on top of the film (n=0) and thus enters in direct contact with the cells. A rapid cellular response is observed already after one hour of contact and it lasts at least up to 6 hours of contact. For the embedded proteins (n=1, 5, 15 and 20), as a general trend, the TNF-α production decreases when the proportion of d peptides in the film increases. More precisely, when x equals 50% or more, the activity is totally suppressed even when PA is embedded under a (PL/PGA/PL) trilayer. For x lying between 0 and 40%, the biological activity can be finely tuned in time with a precision of the order of 1 hour by adjusting both the d polypeptide content and the embedding depth of the protein. More precisely, for x ranging between 30 and 40%, the activity gradually increases after 2 hours of contact when PA is embedded under 1 layer pair. When embedded under 5 pairs of layers, the activity of the PA is totally suppressed during the first 4 hours, whereas the protein becomes fully active after 6 hours of contact. Finally, when embedded under 15 pairs of layers or more, PA no longer acts on cells within the first 6 hours of contact. For the d content ranging between 10 and 20%, full activity is observed for all embedding depths up to n=20 after 4 hours of contact between the cells and the film. For embedding depths up to 5 pairs of layers the activity increases continuously in time, the plateau value being reached after 4 hours of contact. Finally, when the embedding depth exceeds 15 pairs of layers, no biological response takes place during the initial 3 hours of contact but reaches its full activity after 4 hours of contact. This behavior is thus similar to the one found for x ranging from 30 to 40% and an embedding depth of 5 pairs of layers but it takes place at shorter times and larger embedding depths. These results clearly demonstrate that the biological activity of the films can be finely tuned at a time scale of about 1 hour and over at least 6 hours. A continuously increasing activity starting as soon as the cells are brought in contact with the film can be obtained by embedding the proteins under a small number of layer pairs. An “off-on” response in time is seen for higher embedding depths, the time at which the biological response turns on becoming increased with the d content of the solution (consequently also of the film architecture). Now the inventors come back to the total suppression of the biological activity when protein A is embedded under a PL/PGA/PL layer with x=100% or 50%. This unexpected result can be either due to the fact that the protein A molecules are entirely wrapped with polypeptides, which prevent, for high d content, any interaction between protein A and cells or it can be due to the denaturation of protein A by the d polypeptides present in great proportion. On the other hand, parameters, such as local pH, should not be involved since it is expected that they do not depend on the d/l ratio, two enantiomers being of similar chemical nature. The second hypothesis relative to the protein denaturation can be investigated by comparing the amide-l spectra of protein A interacting with PlL and PdL (FIG. 5) and with PlGA and PdGA. In this latter case, the results are similar to those obtained with poly(lysine). The spectra relative to protein A interacting with PlL and with PdL are indistinguishable. They are also indistinguishable from those obtained by summing the spectra of protein A alone and of PlL (resp. PdL) alone. This clearly indicates that the interactions between protein A and both PlL and/or PdL do not affect the secondary structure of protein A. It is thus expected that it is the wrapping of the proteins by a large proportion of d enantiomers that is responsible for the loss of the biological activity when embedded close to the top of the film.

The inventors have seen that the film thickness depends upon the d/l ratio of the build-up polyelectrolyte solutions and in particular that it decreases when x increases from 0 up to 50%. On the other hand, for a given number of deposited pairs of layers, the biological activity decreases when x increases. The variations of the biological effects can thus not be attributed to changes of the film thickness with x. The biological effects can rather be explained by a continuous degradation and pseudopod development through the multilayered films^{[19]} as already found on (PlL/PlGA)ₙ films. One can point out that the film degradation does not take place in the sole presence of culture medium but requires the presence of macrophages. Indeed, images of films in the presence and absence of culture medium were taken by CLSM and no structural changes could be observed as they were observed in the presence of macrophages (FIG. 2a versus 2c). For high d contents (x≧50%), this degradation and pseudopod development are not possible within the first 6 hours. This is confirmed by CLSM observations in which films containing 50% d enantiomers did not show any degradation after even 8 hours of contact and looked similar to films constructed with pure d solutions, i.e. containing exclusively PdGA and PdL (FIG. 1d). For small d contents (x≦20%), the continuous increase of the film activity during the first hours, when PA is embedded under up to 5 pairs of layers, may result from fluctuations in the d and l contents along the multilayer covering the PA layer. This heterogeneity is likely responsible for the high variation in the time needed for the pseudopods to enter in contact with PA. For larger embedding depths, protein A remains out of reach for a period of time which must correspond to the time needed for the cells to degrade the film down to the PA layer. This time is expected to increase with the embedding depth as confirmed by our observations. In contrast, what is unexpected is the rapid “off-on” switch effect which takes place over a typical lapse time of the order of one hour, and which is observed when the proteins are embedded under a large number of pairs of layers for x=10% and 20% and under 5 pairs of layers for x=30 and 40%. This observation could be the consequence of smaller fluctuations of the local d content when the multilayered films are constituted by more than 10 pairs of layers or when the d content is high. Moreover, it must also be a consequence of the well defined depth at which the proteins are embedded. This “off-on” switch effect constitutes a very valuable tool for a fine tuning of the activity of multifunctional films. One can also notice that, by analogy with PlL/PlGA films, both polylysine and polyglutamic acid should diffuse in and out of the entire multilayer film during its construction. There might be some influence of the diffusion of PlL and PlGA on the film degradation. Indeed, degradation of PlL (or PlGA) could lead to the formation of PlL (resp. PlGA) chains of smaller mass. They could then eventually exchange chains of similar nature but of higher mass in the film and thus participate to some extent in the film degradation.

In conclusion, the inventors developed a tool to build up predefined time scheduled bioactive films. This is achieved by taking advantage of the great flexibility offered by the layer-by-layer deposition technology. The multilayer films were constructed using mixtures of degradable and non-degradable polyelectrolyte solutions of known compositions. The inventors took also advantage of the possibility to incorporate the proteins at a very well defined depth in the film with a precision of a few tens of nanometers.

### REFERENCES

[1] I. DeScheerder, K. Wang, K. Wilczek, D. Meuleman, R. VanAmsterdam, G. Vogel, J. Piessens, F. Van de Werf. Circulation 1997, 95, 1549
[2] C. Hehrlein, A. Arab, C. Bode. Basic Research in Cardiology 1997, 97, 417
[3] W. L. Murphy, M. C. Peters, D. H. Kohn and D. J. Mooney. Biomaterials 2000, 21, 2521
[4] D. Arcos, C. V. Ragel and M. Vallet-Regi. Biomaterials 2001, 22, 701
[5] C. T. Laurencin, M. A. Attawia, L. Q. Lu, M. D. Borden, H. H. Lu, W. J. Gorum, J. R. Liebemman. Biomaterials 2001, 22, 1271
[6] Y. Lvov, H. Haas, G. Decher, H. Möhwald, A. Mikhailov, B. Mtchedlishvily, E. Morgunova, B. Vainstein. Langmuir 1994, 10, 4232
[7] Y. Lvov, K. Ariga, I. Ichinose, T. Kunitake. J. Am. Chem. Soc. 1995, 117, 6117
[8] W. Kong, L. P. Wang, M. L. Gao, H. Zhou, X. Zhang, W. Li, J. C. Shen. J. Am. Chem. Soc. 1994, 1297
[9] K. C. Dee, T. T. Andersen, R. Bizios J. Biomed. Mater. Res. 1998, 40, 371
[10] J. A. Neff, K. D. Caldwell, P. A. Tresco. J. Biomed. Mater. Res. 1998, 40, 511
[11] S. M. Cannizzaro, R. F. Padera, R. Langer, R. A. Rogers, F. E. Black, M. C. Davies, S. J. B. Tendler, K. M. Shakesheff. Biotechnol. Bioeng. 1998, 58, 529
[12] Y. Ito, J. Zheng, Y. Imanishi, K. Yonezawa, M. Kasuga. Proc. Natl. Acad. Sci. U.S.A. 1996, 93, 3598
[13] Y. Ito, G. Chen and Y. Imanishi. Bioconj. Chem. 1998, 9, 277
[14] A. C. R. Grayson, I. S. Choi, P. P. Wang, H. Brem, M. J. Cima, R. Langer. Nature Materials 2003, 2, 767
[15] G. Decher. Science 1997, 277, 1232
[16] N. Jessel, Ph. Lavalle, F. Meyer, F. Audouin, B. Frisch, P. Schaaf, J. Ogier, G. Decher, J.-C. Voegel. Adv. Mater. 2004, 16, 1507
[17] J. Chluba, J.-C. Voegel, G. Decher, P. Erbacher, P. Schaaf, J. Ogier. Biomacromolecules 2001, 2, 800
[18] N. Jessel, P. Schwinté, P. Falvey, R. Darcy, Y. Haïkel, P. Schaaf, J.-C. Voegel, J. Ogier. Adv. Funct. Mater. 2004, 14, 174
[19] N. Jessel, F. Atalar, Ph. Lavalle, J. Mutterer, G. Decher, P. Schaaf, J.-C. Voegel, J. Ogier. Adv. Mater. 2003, 15(9), 692
[20] J. D. Mendelsohn, S. Y. Yang, J. Hiller, A. I. Hochbaum, M. F. Rubner. Biomacromolecules 2003, 4, 96
[21] Y. Lvov, H. Haas, G. Decher, H. Möhwald, A. Mikhailov, B. Mtchedlishvily, E. Morgunova, B. Vainstein. Langmuir 1994, 10, 4232
[22] W. Kong, L. P. Wang, M. L. Gao, H. Zhou, X. Zhang, W. Li, J. C. Shen. J. Chem. Soc. Chem. Commun. 1994, 1297
[23] G. Ladam, P. Schaaf, F. Cuisinier, G. Decher, J.-C. Voegel. Langmuir 2001, 17, 878
[24] F. Caruso, K. Niikura, D. N. Furlong, Y. Okahata. Langmuir 1997, 13, 3427
[25] P. Schwinté, J.-C. Voegel, C. Picart, Y. Haikel, P. Schaaf, B. Szalontai. J. Phys. Chem. B. 2001, 105, 11906
[26] S. Kumar, Y. Shukla, A. K. Prasad, A. S. Verma, P. D. Dwividi, N. K. Mehrotra, P. K. Ray. Cancer Lett. 1992, 61, 105
[27] A. S. Verma, P. D. Dwivedi, A. Mishra, P. K. Ray. Immunopharmacol. Immunotoxicol. 1999, 21, 89
[28] V. Subbulakshmi, A. K. Ghosh, T. Das, P. K. Ray. Biochem. Biophys. Res. Comm. 1998, 2, 50.
[29] P. K Ray, S. Raychaudhury, P. Allen. Cancer Res. 1982, 42, 4970
[30] A. K. Srivastava, K. P. Singh, P. K. Ray. Mycopathologia 1997, 138, 21
[31] A. C. Ghose, A. Mookerjee, K. Sengupta, A. K. Ghosh, S. Dasgupta, P. K. Ray. Immunol. Lett. 1999, 65, 175
[32] M. Frankenberger, H. Pechumer, H. W. Ziegler-Heitrock. J. Inflamm. 1995, 45, 56
[33] S. Leporatti, C. Gao, A. Voight, E. Donath, H. Möhwald. Eur. Phys. J 2001, 5, 13
[34] Z. Sui, J. B. Schlenoff. Langmuir 2003, 19, 7829
[35] M. Debreczeny, V. Ball, F. Boulmedais, B. Szalontai, J.-C. Voegel, P. Schaaf. J. Phys. Chem. B 2003, 107, 12734
[36] E. Hüibsch, V. Ball, B. Senger, G. Decher, J.-C. Voegel, P. Schaaf. Langmuir 2004, 20, 1980
[37] M. V. Voinova, M. Rodahl, M. Jonson, B. Kasemo. Physica Scripta 1999, 59, 391
[38] F. Boulmedais, M. Bozonnet, P. Schwinté, J.-C. Voegel, P. Schaaf. Langmuir 2003, 19, 9873
[39] L. Richert, Ph. Lavalle, D. Vautier, B. Senger, J.-F. Stoltz, P. Schaaf, J.-C. Voegel, C. Picart. Biomacromolecules 2002, 3, 1170
[40] P. K. Lund, G. B. Joo, A. B. Westvik, R. Ovstebo, P. Kierulf. J. Immunol. Methods. 1988, 112, 213
[41] J. D. Mendelsohn, C. J. Barrett, V. V. Chan, A. J. Pal, A. M. Mayes, M. F. Rubner. Langmuir 2000, 16, 5017
[42] S. T. Dubas, J. B. Schlenoff. Langmuir 2001, 17, 7725
[43] S. T. Dubas, J. B. Schlenoff. Macromolecules 2001, 34, 3736

1-19. (canceled)

20. A polyelectrolyte multilayer film, wherein said film comprises at least one layer pair of cationic polypeptides and anionic polypeptides and at least one positively and/or negatively charged biological active ingredient, and said cationic polypeptides comprise l and d amino-acid forms and said anionic polypeptides comprise l and d amino-acid forms.

21. The film according to claim 20, wherein the cationic polypeptides are selected in the group consisting of poly(lysine), poly(arginine), poly(ornithine), poly(histidine) and mixtures thereof or more generally of any kind of l and d forms of cationic polypeptides.

22. The film according to claim 20, wherein the anionic polypeptides are selected in the group consisting of poly(glutamic acid), poly(aspartic acid) and mixtures thereof or more generally of any kind of l and d forms of anionic polypeptides.

23. The film according to claim 20, wherein cationic polypeptides and anionic polypeptides are respectively poly(lysine) and poly(glutamic acid).

24. The film according to claim 20, wherein the positively and/or negatively charged biological active ingredient is selected in the group consisting of synthetic polyions (polymers presenting ions), biopolymers such as DNA, RNA, collagen, peptides (such as a RGD sequence, Melanoma stimulating Hormone, or buforin), proteins, growth factors, and enzymes, cells, viruses, dendrimers, colloids, inorganic and organic particles, dyes, vesicles, nano(or micro)capsules, nano(or micro)particles, polyelectrolytes complexes, free or complexed drugs, cyclodextrins, and mixtures thereof.

25. The film according to claim 20, wherein the number of said layer pairs is from 1 to 1000, preferably from 2 to 100, more preferably from 5 to 60.

26. The film according to claim 20, wherein it comprises (1) a first polyelectrolyte multilayer film, said first film (or precursor film) comprising at least one positively and/or negatively charged biological active ingredient and at least one, preferably five, layer pair of cationic polypeptides and anionic polypeptides, said polypeptides presenting only l amino-acid form, and (2) a second polyelectrolyte multilayer film comprising at least one layer pair of cationic polypeptides and anionic polypeptides and wherein each cationic or anionic polypeptide layer includes l and d amino-acid forms.

27. The film according to claim 20, wherein the percentage of l and d amino-acid forms in the cationic polypeptide layer is the same as the percentage of l and d amino-acid forms in the anionic polypeptide layer.

28. The film according to claim 20, wherein the weight percentage x % of d amino-acid form present in the polypeptides of the multilayer film is from 0.1 to 50%, more preferably from 10 to 40%, and more preferably from 20 to 40%.

29. The film according to claim 26, wherein the number of layer pairs in the first film is from 1 to 1000, preferably from 2 to 100, more preferably from 5 to 60.

30. The film according to claim 26, wherein the number of layer pairs in the second film is from 1 to 100, preferably from 2 to 100, more preferably from 20 to 60.

31. A method of coating a surface, wherein said method comprises (1) sequentially depositing on a surface alternating layers of polyelectrolytes to provide a coated surface, wherein a first (or conversely second) polymer is a cationic polypeptide and a second (or conversely first) polymer is an anionic polypolypeptide, said cationic polypeptides comprise l and d amino-acid forms and said anionic polypeptides comprise l and d amino-acid forms polypeptides.

32. The method according to claim 31, wherein said method further comprises (2) reacting a surface with a solution comprising at least one positively and/or negatively charged biologically active ingredient.

33. The method according to claim 31, wherein said surface, before step (1), is a surface coated by a first film (or precursor film) comprising at least one layer pair of cationic polypeptides and anionic polypeptides, said polypeptides presenting only l amino-acid forms, and optionally at least one positively and/or negatively charged biological active ingredient.

34. The method according to claim 31, wherein depositing on a surface alternating layers of polypolypeptides includes dipping, dip-coating, rinsing, dip-rinsing, spraying, inkjet printing, stamping, printing and microcontact printing, wiping, doctor blading or spin coating.

35. The method according to claim 31, wherein depositing on a surface alternating layers of polypolypeptides involves coating and rinsing steps.

36. A coated article obtained by a method according to claim 31.

37. The coated article according to claim 36, wherein the coating comprises a polyelectrolyte multilayer film, wherein said film comprises at least one layer pair of cationic polypeptides and anionic polypeptides and at least one positively and/or negatively charged biological active ingredient, and said cationic polypeptides comprise l and d amino-acid forms and said anionic polypeptides comprise l and d amino-acid forms.

38. The coated article according to claim 36, wherein said article is selected from the group consisting of blood vessel stents, angioplasty balloons, vascular graft tubing, prosthetic blood vessels, vascular shunts, heart valves, artificial heart components, pacemakers, pacemaker electrodes, pacemaker leads, ventricular assist devices, contact lenses, intraocular lenses, sponges for tissue engineering, foams for tissue engineering, matrices for tissue engineering, scaffolds for tissue engineering, biomedical membranes, dialysis membranes, cell-encapsulating membranes, drug delivery reservoirs, drug delivery matrices, drug delivery pumps, catheters, tubing, cosmetic surgery prostheses, orthopedic prostheses, dental prostheses, bone and dental implant, wound dressings, sutures, soft tissue repair meshes, percutaneous devices, diagnostic biosensors, cellular arrays, cellular networks, microfluidic devices, and protein arrays.
